(19) United States
(12) Patent Application Publication
(10) Pub. No.: US 2017/0223969 A1
(43) Pub. Date: Aug. 10, 2017

(54) ANTIMICROBIAL COMPOSITIONS FROM PRUNUS

(71) Applicant: Branch Research LLC, Chantilly, VA (US)

(72) Inventor: Donald Roberts, Salem, OR (US)

(21) Appl. No.: 15/422,799

(22) Filed: Feb. 2, 2017

Related U.S. Application Data
(60) Provisional application No. 62/291,555, filed on Feb. 5, 2016.

(51) Int. Cl. A01N 65/34; A61K 36/736

(52) U.S. Cl. CPC A01N 65/34; A61K 36/736

(57) ABSTRACT

The foliage and stems of plant species from the family Rosacea, genus Prunus, yield natural pesticides when macerated. Hydrodistillation of macerated plant biomass yields a concentrated solution of organic volatile compounds that act synergistically as a natural pesticide, and as provided herein, also act synergistically as antimicrobial compounds. Volatile compounds liberated from Prunus biomass include 2-propanol, hexanal, trans-2-hexenal, 1-hexanol, cis-3-hexenol, mandelonitrile, benzoic acid, benzaldehyde, benzyl alcohol, hydrocyanic acid and others. These compounds may be removed from the distillate and reformulated to form a standard concentrated solution, with benzaldehyde, mandelonitrile and hydrogen cyanide being the major components. Provided herein are methods of using these pesticides as a broad-spectrum bactericide. Components of the extract may act alone or synergistically to control both gram positive and gram negative genera of bacteria.

### CROSS-REFERENCE TO RELATED APPLICATIONS

This application claims the benefit of U.S. application Ser. No. 62/291,555, filed Feb. 5, 2016. This application is incorporated herein by reference in its entirety.

### FIELD OF THE INVENTION

The invention generally relates to compounds and compositions produced from the biomass of plants that exhibit antimicrobial, bactericidal, and fungicidal properties.

### BACKGROUND OF THE INVENTION

Bacterial infections are a major health problem and pose a large economic burden on our society. Bacterial infections can be caused by a wide range of bacteria, resulting in mild to life-threatening illnesses that require immediate intervention. Common bacterial infections include for example pneumonia, ear infections, diarrhea, urinary tract infections, and skin disorders.

Bacterial infections also constantly threaten reduction of the value of ornamental plants and cause crop losses. Unabated, diseases may significantly reduce plant survival, vigor, and yield. Synthetic chemicals are typically used to control such infections around homes, and in the agricultural and horticultural industries. However, as the general population and regulatory authorities become more sensitive to the health hazards of synthetic chemicals, more interest and effort in finding safe substitutes are evident.

Moreover, it is becoming more evident that antibiotic compositions do not always perform to the extent they should. Some infections cannot be cleared, even if the pathogen is sensitive to the used antibiotic. This inability to completely kill all bacteria poses a severe problem once the antibiotic treatment is stopped, as the infection relapses. Moreover, the constant antibiotic pressure and the natural competence of some strains results in frequent resistance to antibiotics. Thus, there is a need for the development of new antibacterial compositions that avoid the hazards of synthetic chemicals.

### SUMMARY OF THE INVENTION

One aspect of the invention provides antibacterial compounds and compositions obtained from plants in the family Rosaceae, genus Prunus. Volatile compounds liberated from Prunus biomass including 2-propanol, hexanal, trans-2-hexenal, 1-hexanol, cis-3-hexenol, mandelonitrile, benzoic acid, benzaldehyde, benzyl alcohol, hydrocyanic acid and others were previously found to act as an effective natural pesticide (see U.S. Pat. No. 7,195,788 herein incorporated by reference). However, the antimicrobial (e.g., antibacterial and antifungal) properties of these compounds were not previously explored.

In some embodiments, the invention provides a substrate having a composition impregnated within or carried on said substrate, wherein said composition is obtained from a Prunus biomas. In some embodiments, the composition comprises two or more compounds selected from the group consisting of 2-propanol, hexanal. trans-2-hexenal, 1-hexanol, cis-3-hexenol, trans-2-hexenol, mandelonitrile, benzoic acid, benzaldehyde, benzyl alcohol, and hydrocyanic acid. Exemplary substrates may be composed of paper, cloth, or a sponge.

In other embodiments, the invention provides a method for inhibiting or slowing growth of microbes (e.g., bacteria or fungi), comprising contacting a surface with a substrate of the invention (e.g., wiping a counter with a paper or cloth substrate including hydrodistillate products of Prunus biomass, e.g., two or more of 2-propanol, hexanal, trans-2-hexenal, 1-hexanol, cis-3-hexenol, trans-2-hexenol, mandelonitrile, benzoic acid, benzaldehyde, benzyl alcohol, and hydrocyanic acid). Inhibiting or slowing the growth of microbes such as bacteria and fungi may also be achieved by other means of bringing the hydrodistillate products of Prunus biomass into contact with the microbes (e.g., spraying on surfaces on which the bacteria are known to be located; adding to fluids or other materials in which the bacteria are known to be located, etc.). Other embodiments of the invention provide a method for controlling growth (e.g., killing, inhibiting or slowing growth, etc.) of microbes (e.g., bacteria or fungi), comprising the steps of identifying a subject (e.g., plant or animal) having a bacterial infection; and contacting the microbes causing said infection with a composition obtained from a Prunus biomass. In some embodiments, the composition comprises two or more compounds selected from the group consisting of 2-propanol, hexanal, trans-2-hexenal, 1-hexanol, cis-3-hexenol, trans-2-hexenol, mandelonitrile, benzoic acid, benzaldehyde, benzyl alcohol, and hydrocyanic acid.

Exemplary bacteria include gram positive bacteria selected from the group consisting of Ralstonia, Agrobacterium, Xanthomonas, Erwinia, Enterococcus, Staphylococcus, Streptococcus, Bacillus, Corynebacterium, and Listeria and gram negative bacteria selected from the group consisting of Escherichia, Salmonella, Pseudomonas, and Helicobacter.

### DETAILED DESCRIPTION

Embodiments of the invention relate to novel antimicrobial, bactericidal, and fungicidal applications of a composition containing two or more of 2-propanol, hexenal, trans-2-hexenal, 1-hexanol, cis-3-hexenol, trans-2-hexenol, mandelonitrile, benzoic acid, benzaldehyde, benzyl alcohol, and hydrocyanic acid. Ideally, the compounds in the composition may be obtained from a hydrodistillate of Prunus biomass as is described in U.S. Pat. No. 7,195,788 incorporated herein by reference and/or the concentrated hydrodistillate itself (which would include two or more of the aforementioned compounds).

The compositions contemplated herein have both agricultural and industrial applications. In some embodiments, the compounds of the invention may be incorporated into a wipe (e.g., impregnated or soaked into a paper or cloth or sponge or other suitable substrate) to control or eliminate bacteria such as e. coli and salmonella, for example, in food service areas (e.g., by wiping surfaces contacted by the food or surfaces of the food itself). As an example, in the medical environment, surfaces instruments, bedding, walls, etc. that patients and/or doctors may come into contact may be rendered safer by using the wipe to combat, for example, MERSA. Many compositions, having multiple compounds, show synergistic bactericidal and/or fungicidal activity.

Exemplary new developments include but are not limited to the use of a wipe, having a substrate which can be impregnated with or otherwise carry a composition having two or more compounds selected from the group consisting of 2-propanol, hexenal, trans-2-hexenal, 1-hexanol, cis-3-hexenol, trans-2-hexenol, mandelonitrile, benzoic acid, benzaldehyde, benzyl alcohol, and hydrocyanic acid to combat one or more bacteria by wiping on surfaces of objects to be protected including medical and surgical tools, hospital walls, hospital bedding, door handles, food surfaces, surfaces of containers or tables which come in contact with food, etc. This use may be accomplished with a hydrodistillate of Prunus biomass.

An additional application involves the use of a composition having two or more compounds selected from the group consisting of 2-propanol, hexenal, trans-2-hexenal, 1-hexanol, cis-3-hexenol, trans-2-hexenol, mandelonitrile, benzoic acid, benzaldehyde, benzyl alcohol, and hydrocyanic acid to combat growth of one or more bacteria in food processing applications, or harvesting applications, or in the field during growth phase of plants that produce the food products. This use may be accomplished with a hydrodistillate of Prunus biomass.

The compositions may be obtained from the reproductive and/or vegetative biomass of Prunus species. Representative compositions are extracted from the vegetative biomass of plants of the genus Prunus. In a particular embodiment, the vegetative biomass comprises Prunus stems and leaves. Biomass may be obtained from most Prunus species, including P. serotina, P. virginiana, P. emarginata, P. dulcis, and P. persica, cultivars and varieties thereof, and combinations thereof.

The compositions of the invention are effective against both gram positive and gram negative genera of bacteria. Exemplary gram positive bacteria include, but are not limited to, Ralstonia, Agrobacterium, Xanthomonas, Erwinia, Enterococcus, Staphylococcus, Streptococcus, Bacillus, Corynebacterium, and Listeria. Exemplary gram negative bacteria, include, but are not limited to, Escherichia, Salmonella, Pseudomonas, and Helicobacter. The compositions may also be used for controlling other microbes such as fungi

The composition of the invention may be administered to any subject, for example, mammalian subjects including humans or nonanimal subjects such as plants. The compositions may be applied directly or indirectly topically to the subject. The subject may be afflicted with or susceptible to be afflicted with a bacterial or other microbial infection.

Embodiments of the invention provide methods for controlling, preventing, treating or curing bacterial or other microbial infections affecting plants. In one embodiment, plants are exposed to Prunus extracts or a composition containing two or more of 2-propanol, hexenal, trans-2-hexenal, 1-hexanol, cis-3-hexenol, trans-2-hexenol, mandelonitrile, benzoic acid, benzaldehyde, benzyl alcohol, and hydrocyanic acid (e.g. by contact) to control plant pests and pathogens. In a particular embodiment, a method for controlling growth (e.g., killing or inhibiting growth of) of a pathogenic organism is provided wherein the pathogenic organism is exposed to a composition obtained from Prunus biomass or a composition containing two or more of 2-propanol, hexenal, trans-2-hexenal, 1-hexanol, cis-3-hexenol, trans-2-hexenol, mandelonitrile, benzoic acid, benzaldehyde, benzyl alcohol, and hydrocyanic acid. Exposure of the pathogenic microorganism to at least a component of the composition is maintained for a time sufficient to control the growth of the pathogenic microorganism (e.g., to kill or reduce or otherwise inhibit its growth). In other particular embodiments, Prunus extracts or a composition containing two or more of 2-propanol, hexenal, trans-2-hexenal, 1-hexanol, cis-3-hexenol, trans-2-hexenol, mandelonitrile, benzoic acid, benzaldehyde, benzyl alcohol, and hydrocyanic acid are applied foliarly or directly to soil to control a disease or infestation. Extracts may also be applied to soil as fumigants or as a drench, and to foliage as a spray, mist, or drench. Prunus extracts or a composition containing two or more of 2-propanol, hexenal, trans-2-hexenal, 1-hexanol, cis-3-hexenol, trans-2-hexenol, mandelonitrile, benzoic acid, benzaldehyde, benzyl alcohol, and hydrocyanic acid may be applied to control fungal and bacterial diseases.

Stored commodities such as fruit, vegetables, roots, grains and nuts may be treated with Prunus extracts or a composition containing two or more of 2-propanol, hexenal, trans-2-hexenal, 1-hexanol, cis-3-hexenol, trans-2-hexenol, mandelonitrile, benzoic acid, benzaldehyde, benzyl alcohol, and hydrocyanic acid as a in a spray, drench or fumigant to protect them from fungal infections and insect infestations to control post-harvest losses. Prunus extracts or a composition containing two or more of 2-propanol, hexenal, trans-2-hexenal, 1-hexanol, cis-3-hexenol, trans-2-hexenol, mandelonitrile, benzoic acid, benzaldehyde, benzyl alcohol, and hydrocyanic acid may be applied as an aqueous spray or may be made into a solution with an ionic, non-ionic or cationic emulsifier, such as any surfactant having an hydrophile-lipophile balance (HLB, see, for example, W. C. Griffin, J. Soc. Cosmetic Chemicals, 1: 311, 1949) of 1-20, and then applied as a spray. Prunus extracts or a composition containing two or more of 2-propanol, hexenal, trans-2-hexenal, 1-hexanol, cis-3-hexenol, trans-2-hexenol, mandelonitrile, benzoic acid, benzaldehyde, benzyl alcohol, and hydrocyanic acid may also be spray-dried and applied as a powder, for example, by incorporating the extracts into a substrate such as maltodextrin, dextrin, or disaccharide. The Prunus extracts or a composition containing two or more of 2-propanol, hexenal, trans-2-hexenal, 1-hexanol, cis-3-hexenol, trans-2-hexenol, mandelonitrile, benzoic acid, benzaldehyde, benzyl alcohol, and hydrocyanic acid may also be incorporated in a coating or wax used to treat foods or post-harvest commodities. Prunus extracts or a composition containing two or more of 2-propanol, hexenal, trans-2-hexenal, 1-hexanol, cis-3-hexenol, trans-2-hexenol, mandelonitrile, benzoic acid, benzaldehyde, benzyl alcohol, and hydrocyanic acid may be used for disinfection by fumigation or drench of packaging or enclosed containers that will contain foods, such as harvest bins, storage chambers and shipping containers. Fumigation may, for example, be accomplished by spraying an aqueous solution or powder, by vaporization of extracts, or by incorporating the extracts into a time-release device such as by encapsulation in a polymer matrix, polyvinylchloride strip, or rubber pellets.

Compositions comprising Prunus macerate slurries, macerate solutions, hydrodistillates, and concentrated extracts may be formulated with various adjuvants to aid in their stability and effectiveness as bactericides or microbiocides. Adjuvants include emulsifiers, surfactants (including non-ionic surfactants, cationic surfactants and anionic surfactants), stickers, stabilizers, penetrants, extenders, and corrosion inhibitors. For example, concentrated extracts may be made more water-soluble by adding of emulsifiers such as polysorbate 80. Emulsifiers include polyethyleneoxide derivatives (e.g., TWEEN® 20 series). Soil treatments comprising Prunus compositions may benefit from formulation with a soil surfactant, such as ammonium laureth sulfate, sodium laureth sulfate, nonyl phenol ethoxylate, or combination thereof. When Prunus compositions are applied foliarly it may be desirable to add non-ionic surfactants, such as octylphenol compounds or ethylated seed oils plus emulsifiers, to enhance spreading of the compositions on surfaces. Extenders, such as blends of organic silicone and seed oils, may be desirable to reduce volatilization of composition constituents. Likewise, addition of penetrants, such as ethoxylated and linear alcohols combined with silicone or organic silicone and esterified seed oils, desirably assist movement of the compositions into the pores of the target plant tissue.

An additional aspect of the invention relates to methods of administering a composition as described herein to a mammalian subject, such as a human. The methods involve administering an effective amount of the composition, where the effective amount is sufficient to prevent the development of or lessen the extent of the development of a bacterial or fungal infection in the subject, when the subject is later exposed to the bacterium or contracts a bacterial infection.

The compositions provided herein can be administered using any appropriate method. Administration can be, for example, topical (e.g. transdermal, ophthalmic or intranasal); pulmonary (e.g., by inhalation or insufflation or powders or aerosols); oral, or parenteral (e.g. by subcutaneous, intrathecal, intraventricular, intramuscular, or intraperitoneal injection, or by intravenous drip).

The compositions of the invention can include a pharmacologically suitable vehicle or carrier. The preparation of such compositions for use as vaccines is well known to those of skill in the art. Typically, such compositions are prepared either as liquid solutions or suspensions, however solid forms such as tablets, pills, powders and the like are also contemplated. The liquid may be an aqueous liquid. Solid forms suitable for solution in, or suspension in, liquids prior to administration may also be prepared. The preparation may also be emulsified. The active ingredients may be mixed with excipients, which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. The compositions of the present invention may further comprise an adjuvant, suitable examples of which include but are not limited to Seppic, Quil A, Alhydrogel, etc. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of active agents in the formulations may vary. However, in general, the amount in the formulations will be from about 0.01-99%, weight/volume.

In addition, the compositions may be administered in conjunction with other treatment modalities such as substances that boost the immune system, antibiotics, and the like.

The term “treating” or “treatment”, as used herein, means reversing, alleviating, inhibiting the progress of, or preventing the condition to which such term applies, or one or more symptoms of such disorder or condition. A “therapeutically effective amount” is intended for a minimal amount of active agent which is necessary to impart therapeutic benefit to a subject. For example, a “therapeutically effective amount” to a mammal is such an amount which induces, ameliorates or otherwise causes an improvement in the pathological symptoms, disease progression or physiological conditions associated with or resistance to succumbing to an infection.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLE 1

Premier Botanicals, Ltd supplied two formulation of the Prunus extract to an analytical laboratory to conduct a Biocide Efficacy Trial against bacterial species representing Gram negative and gram positive type. Formulation identified as ‘Extraction #2’ is an essential oil obtained by hydrodistillation of plant material collected from the Prunus plants. Formulation identified as ‘Extraction #3’ is a redistilled oil from ‘Extraction #2’.

The results of the test are presented below.

Sample Description:

The samples consist of two proprietary formulations from Premier Botanicals as follows:
001. Extraction #2
002. Extraction #3

The objective of analysis is to test samples 001 and 002 as follows:
I. Challenge testing to determine efficacy of biocide in inhibiting growth of selected microorganisms.

Analytic Procedure:

I. Challenge Testing

The following microorganisms were used in this challenge test:

1. Escherichia coli. ATCC 51813.

2. Salmonella abaetetuba. ATCC 35640.

3. Listeria innocua. ATCC 33090.

Protocol:
1. Each of the 3 microorganisms were streaked on Standard Methods Agar (SMA) slants and incubated at 35° C. for 24 hrs. Each culture was washed off the agar slants and serially diluted until a concentration of approximately 300-500 Colony Forming Units (CFU)/ml was reached.
2. Each of the 2 client formulations and a control sample were prepared using a Gatorade/Water mix (50:50 v/v) to achieve the following concentrations:
   001:0.5%
   002: 0.5%
   Control: No Biocide
3. 9.0 ml of each formulation and control was aseptically transferred to a set of 3 sterile tubes.
4. Each of the 3 tubes were challenged with 1.0 ml of a different microorganism and vortexed to mix thoroughly so that each formulation was challenged with all 3 microorganisms.
5. Challenged samples were kept at room temperature (approximately 25° C.) for a total of 2 hrs. 1 ml of each sample was plated onto SMA at 0 and 120 minutes. The original inoculum was also plated at 0 and 120 minutes for comparison. Inoculated plates were incubated at 35° C. for 48 hrs.
6. The effect of the biocide on bacteria exposed to the formulations was assessed by comparing approximate counts of sample plates to the approximate count of the initial inoculum following incubation.

### RESULTS

I. Challenge Testing

| | | | | | |
|---|---|---|---|---|---|
| | Time | | ~CFU | ~CFU | % |
| Microorganism | (m) | Concentration | Inoculum | Sample | Inhibition |

001: Extract #2

| | | | | | |
|---|---|---|---|---|---|
| E. coli | 0 | 0.5% | 35 | 0 | 100 |
| | 120 | 0.5% | 35 | 0 | 100 |
| S. abaetetuba | 0 | 0.5% | 25 | 0 | 100 |
| | 120 | 0.5% | 25 | 0 | 100 |
| L. innocua | 0 | 0.5% | 40 | 0 | 100 |
| | 120 | 0.5% | 40 | 0 | 100 |

002: Extract #3

| | | | | | |
|---|---|---|---|---|---|
| E. coli | 0 | 0.5% | 35 | 0 | 100 |
| | 120 | 0.5% | 35 | 0 | 100 |
| S. abaetetuba | 0 | 0.5% | 25 | 0 | 100 |
| | 120 | 0.5% | 25 | 0 | 100 |
| L. innocua | 0 | 0.5% | 40 | 0 | 100 |
| | 120 | 0.5% | 40 | 0 | 100 |
| E. coli | 0 | N/A | 35 | 35 | 0 |
| | 120 | N/A | 35 | 30 | 15 |
| S. abaetetuba | 0 | N/A | 25 | 25 | 0 |
| | 120 | N/A | 25 | 25 | 0 |
| L. innocua | 0 | N/A | 40 | 40 | 0 |
| | 120 | N/A | 40 | 35 | 12 |

### DISCUSSION

As the data demonstrates, both formulations at the tested concentrations effectively inhibited growth of all the selected microorganisms, both initially and over the duration of the test period. Any reduction of microorganisms in the Control sample may be attributed to die-off or variations in the plated aliquot.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A substrate having a composition impregnated within or carried on said substrate, wherein said composition is or is obtained from a hydrodistillate of Prunus biomass.

2. The substrate of claim 1, wherein said substrate is composed of paper, cloth, or a sponge

3. A substrate having a composition impregnated within or carried on said substrate, wherein said composition includes two or more compounds selected from the group consisting of 2-propanol, hexanal, trans-2-hexenal, 1-hexanol, cis-3-hexenol, trans-2-hexenol, mandelonitrile, benzoic acid, benzaldehyde, benzyl alcohol, and hydrocyanic acid.

4. The substrate of claim 3, wherein said substrate is composed of paper, cloth, or a sponge.

5. A method for inhibiting or slowing growth of microbes, comprising contacting a surface with a substrate according to claim 3.

6. The method of claim 5 wherein the microbes are bacteria.

7. The method of claim 6 wherein said bacteria is a gram-positive bacterium selected from the group consisting of Ralstonia, Agrobacterium, Xanthomonas, Erwinia, Enterococcus, Staphylococcus, Streptococcus, Bacillus, Corynebacterium, and Listeria.

8. The method of claim 6 wherein said bacteria is a gram-negative bacterium selected from the group consisting of Escherichia, Salmonella, Pseudomonas, and Helicobacter.

9. A method for controlling growth of bacteria, comprising the steps of:
identifying a subject having a bacterial infection; and
contacting the bacteria causing said infection with a composition which is or is obtained from a hydrodistillate of Prunus biomass, or which comprises two or more compounds selected from the group consisting of 2-propanol, hexanal, trans-2-hexenal, 1-hexanol, cis-3-hexenol, trans-2-hexenol, mandelonitrile, benzoic acid, benzaldehyde, benzyl alcohol, and hydrocyanic acid.

10. The method of claim 9 wherein said bacteria is a gram-positive bacterium selected from the group consisting of Ralstonia, Agrobacterium, Xanthomonas, Erwinia, Enterococcus, Staphylococcus, Streptococcus, Bacillus, Corynebacterium, and Listeria.

11. The method of claim 9 wherein said bacteria is a gram-negative bacterium selected from the group consisting of Escherichia, Salmonella, Pseudomonas, and Helicobacter.

12. The method of claim 9 wherein said subject is a human.

13. The method of claim 9 wherein said subject is a plant.

14. A method for controlling microbial growth comprising the step of contacting microbes with a composition which is or is obtained from a hydrodistillate of Prunus biomass, or which comprises two or more compounds selected from the group consisting of 2-propanol, hexanal, trans-2-hexenal, 1-hexanol, cis-3-hexenol, trans-2-hexenol, mandelonitrile, benzoic acid, benzaldehyde, benzyl alcohol, and hydrocyanic acid.

15. The method of claim 14 wherein said contacting step comprises applying, spraying or drenching a surface of a fruit with the composition.

16. The method of claim 14 wherein said contacting step comprises applying, spraying or drenching a soil with the composition.

17. The method of claim 14 wherein said contacting step comprises applying, spraying or drenching a foliar surface of a plant with the composition.

18. The method of claim 14 wherein said contacting step comprise applying, spraying or drenching a topical surface of an animal with the composition.

19. The method of claim 14 wherein the microbe is selected from bacteria or fungi.
